(19) United States
(12) Patent Application Publication
(10) Pub. No.: US 2013/0019867 A1
(43) Pub. Date: Jan. 24, 2013

(54) ANESTHESIA MACHINE AND SYSTEM

(71) Applicant: James Nyal Mashak, Madison, WI (US)

(72) Inventor: James Nyal Mashak, Madison, WI (US)

(73) Assignee: GENERAL ELECTRIC COMPANY, Schenectady, NY (US)

(21) Appl. No.: 13/186,569

(22) Filed: Jul. 20, 2011

(51) Int. Cl. A61M 16/01; A61M 16/08

(52) U.S. Cl. 128/203.12; 128/200.24

(57) ABSTRACT

An anesthesia machine is disclosed herein in accordance with an embodiment. The anesthesia machine may include a gas mixer, and an anesthesia machine connector pneumatically coupled with the gas mixer. The anesthesia machine connector may be adapted to selectively receive a pneumatic circuit. The anesthesia machine may be operable in a first mode when the pneumatic circuit is decoupled from the anesthesia machine connector. The first mode may comprise the transfer of a gas from the gas mixer to a first destination. The anesthesia machine may be automatically converted to a second mode when the pneumatic circuit is coupled with the anesthesia machine connector. The second mode may comprise the transfer of the gas from the gas mixer to a second destination.

### FIELD OF THE INVENTION

This disclosure relates to an anesthesia machine and system. More precisely, this disclosure relates a system adapted to automatically convert from one operational mode to another when a pneumatic circuit is connected to the anesthesia machine.

### BACKGROUND OF THE INVENTION

Anesthesia may be administered to a patient in the form of a gas to produce an effect such as pain management, unconsciousness, preventing memory formation, and/or paralysis. An anesthesia system may be implemented to deliver a predetermined dosage of anesthetic agent that is inhaled into the patient's lungs to produce one or more of these effects. The anesthesia delivery system may be pneumatically coupled with a patient via a pneumatic circuit.

One problem with conventional systems is that their functionality must generally be manually changed when the pneumatic circuit is connected and disconnected.

### BRIEF DESCRIPTION OF THE INVENTION

The above-mentioned shortcomings, disadvantages and problems are addressed herein which will be understood by reading and understanding the following specification.

In an embodiment, an anesthesia machine includes a gas mixer, and an anesthesia machine connector pneumatically coupled with the gas mixer. The anesthesia machine connector may be adapted to selectively receive a pneumatic circuit. The anesthesia machine may be operable in a first mode when the pneumatic circuit is decoupled from the anesthesia machine connector. The first mode may comprise the transfer of a gas from the gas mixer to a first destination. The anesthesia machine may be automatically converted to a second mode when the pneumatic circuit is coupled with the anesthesia machine connector. The second mode may comprise the transfer of the gas from the gas mixer to a second destination.

In another embodiment, an anesthesia machine includes a gas mixer, and an anesthesia machine connector pneumatically coupled with the gas mixer. The anesthesia machine connector may include an inlet port adapted to receive a gas from the gas mixer, and an outlet port selectively connectable with the inlet port. The outlet port may be adapted to direct the gas to a first destination. The anesthesia machine connector also includes an outlet selectively connectable with the inlet port. The outlet may be adapted to direct the gas to a second destination. The anesthesia machine connector may also include a valve assembly adapted to pneumatically couple the inlet port with the outlet port or the outlet. The valve assembly may couple the inlet port with the outlet port when a pneumatic circuit is disconnected from the anesthesia machine connector. The valve assembly may automatically couple the inlet port with the outlet when the pneumatic circuit is connected to the anesthesia machine connector.

In another embodiment, an anesthesia system may include a pneumatic circuit, and an anesthesia machine. The anesthesia machine may include a gas mixer, and an anesthesia machine connector pneumatically coupled with the gas mixer. The anesthesia machine connector may be adapted to selectively receive the pneumatic circuit. The anesthesia system may also include a scavenging system pneumatically coupled with the anesthesia machine. The anesthesia machine may be operable in a first mode when the pneumatic circuit is decoupled from the anesthesia machine connector. The first mode may comprise the transfer of a gas from the gas mixer to the scavenging system. The anesthesia machine may be automatically converted to a second mode when the pneumatic circuit is coupled with the anesthesia machine connector. The second mode may comprise the transfer of the gas from the gas mixer to a patient.

In another embodiment, a pneumatic circuit may include a tube, and a pneumatic circuit connector secured to the tube. The pneumatic circuit connector may be adapted to selectively couple with an anesthesia machine. The pneumatic circuit connector may be configured to trigger a first anesthesia machine mode when the pneumatic circuit is disconnected from the anesthesia machine. The first anesthesia machine mode comprises the transfer of a gas to a first destination. The pneumatic circuit connector may be configured to trigger a second anesthesia machine mode when the pneumatic circuit is connected to the anesthesia machine. The second anesthesia machine mode comprises the transfer of a gas to a second destination.

Various other features, objects, and advantages of the invention will be made apparent to those skilled in the art from the accompanying drawings and detailed description thereof.

### BRIEF DESCRIPTION OF THE DRAWINGS

FIG. 1 is a schematic representation of an anesthesia system in accordance with an embodiment;

FIG. 2 is a sectional exploded view of a pneumatic circuit and an anesthesia system connector;

FIG. 3 is a sectional view of a pneumatic circuit decoupled from an anesthesia system connector; and

FIG. 4 is a sectional view of a pneumatic circuit coupled with an anesthesia system connector.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

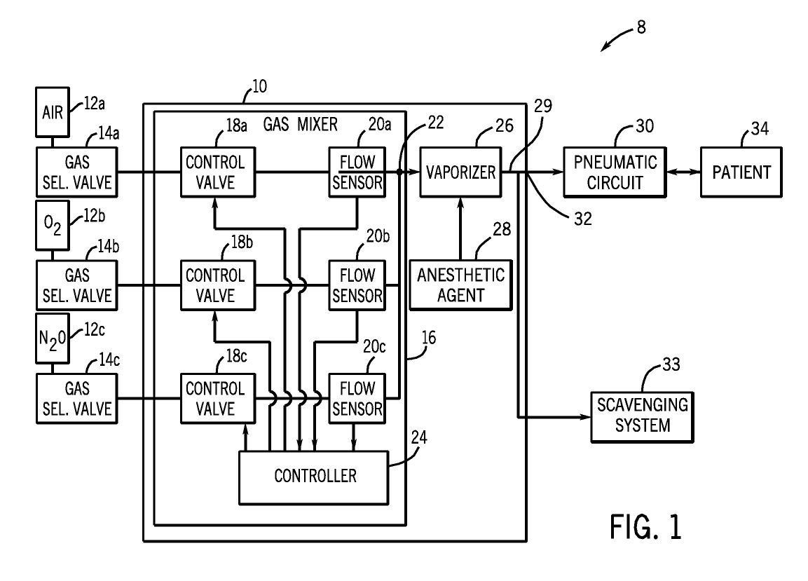

Referring to FIG. 1, an anesthesia system 8 is schematically depicted in accordance with an embodiment. The anesthesia system 8 includes an anesthesia machine 10, a plurality of gas storage devices 12a, 12b and 12c, a plurality of gas selector valves 14a, 14b, and 14c, a pneumatic circuit 30, and an anesthesia machine connector 32. The anesthesia machine 10 is shown for illustrative purposes and it should be appreciated that other types of anesthesia machines may alternately be implemented. In a typical hospital environment, the gas storage devices 12a, 12b and 12c are centrally located storage tanks configured to supply medical gas to multiple anesthesia machines and multiple hospital rooms. The storage tanks are generally pressurized to facilitate the transfer of the medical gas to the anesthesia machine 10.

The gas storage devices 12a, 12b and 12c will hereinafter be described as including an air tank 12a, an oxygen (O2) tank 12b, and a nitrous oxide (N2O) tank 12c, respectively, however it should be appreciated that other storage devices and other types of gas may alternatively be implemented. The gas storage tanks 12a, 12b and 12c are each connected to one of the gas selector valves 14a, 14b, and 14c, respectively. The gas selector valves 14a, 14b and 14c may be implemented to shut off the flow of medical gas from the storage tanks 12a, 12b and 12c when the anesthesia machine 10 is not operational. When one of the gas selector valves 14a, 14b and 14c is opened, gas from a respective storage tank 12a, 12b and 12c is transferred under pressure to the anesthesia machine 10.

The anesthesia machine 10 includes a gas mixer 16 adapted to receive medical gas from the storage tanks 12a, 12b and 12c. The gas mixer 16 includes a plurality of control valves 18a, 18b and 18c that are respectively connected to one of the gas selector valves 14a, 14b and 14c. The gas mixer 16 also includes a plurality of flow sensors 20a, 20b and 20c that are each disposed downstream from a respective control valve 18a, 18b, and 18c. After passing through one of the control valves 18a, 18b and 18c, and passing by one of the flow sensors 20a, 20b and 20c, the individual gasses (i.e., air, O2 and N2O) are combined to form a mixed gas at the mixed gas outlet 22.

The control valves 18a, 18b and 18c and the flow sensors 20a, 20b and 20c are each connected to a controller 24. The controller 24 is configured to operate the control valves 18a, 18b and 18c in a response to gas flow rate feedback from the sensors 20a, 20b and 20c. Accordingly, the controller 24 can be implemented to maintain a selectable flow rate for each gas (i.e., air, O2 and N2O) such that the mixed gas at the mixed gas outlet 22 comprises a selectable ratio of air, O2 and N2O. The mixed gas flows to a vaporizer 26 where an anesthetic agent 28 may be vaporized and added to the mixed gas from the mixed gas outlet 22. The anesthetic agent 28 and/or mixed gas combination is referred to as inhalation gas or fresh gas 29.

The anesthesia machine 10 may include an anesthesia machine connector 32 adapted to receive the pneumatic circuit 30. When the pneumatic circuit 30 is disconnected from the anesthesia machine connector 32, the anesthesia machine 10 may be operable in a first mode in which the fresh gas 29 is directed to a scavenging system 33. The scavenging system 33 may vent the fresh gas 29 outside the room or building in which the anesthesia machine 10 is being implemented. When the pneumatic circuit 30 is connected to the anesthesia machine connector 32, the anesthesia machine 10 may be operable in a second mode in which fresh gas 29 passes through the pneumatic circuit 30 and is delivered to the patient 34. As will be described in more detail, the anesthesia machine 10 is automatically converted from the first mode to the second mode when the pneumatic circuit 30 is connected to the anesthesia machine connector 32. Similarly, the anesthesia machine 10 is automatically converted from the second mode to the first mode when the pneumatic circuit 30 is disconnected from the anesthesia machine connector 32.

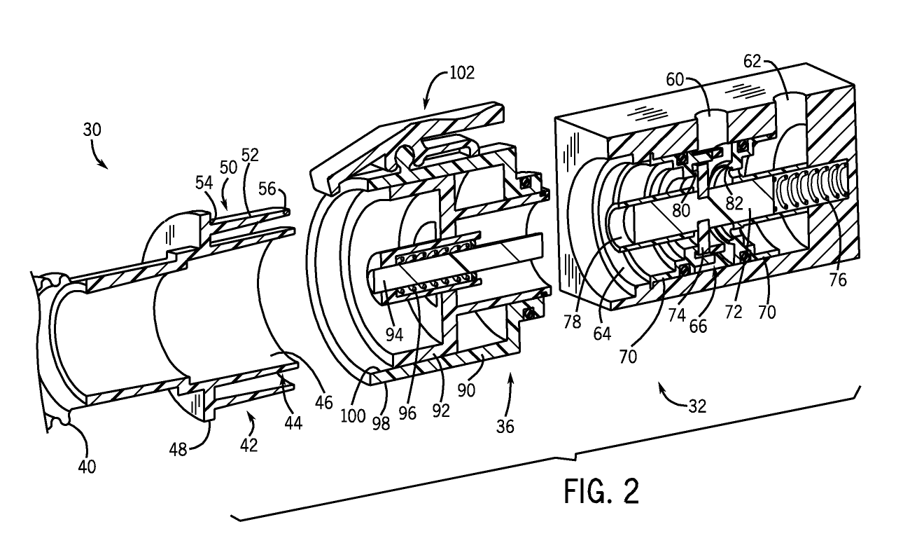

Referring to FIG. 2, the pneumatic circuit 30 and the anesthesia machine connector 32 are shown in accordance with an embodiment. The pneumatic circuit 30 may comprise a tube 40 adapted to direct fresh gas 29 (shown in FIG. 1) from the anesthesia machine 10 (shown in FIG. 1) to a patient. The tube 40 is depicted as comprising corrugated tubing; however other tube configurations may be envisioned. The pneumatic circuit 30 may also comprise a pneumatic circuit connector 42 secured to a terminal end of the tube 40.

The pneumatic circuit connector 42 may comprise a generally cylindrical body 44 defining a hollow interior 46. The hollow interior 46 is in fluid communication with the tube 40 such that the fresh gas 29 (shown in FIG. 1) is transferrable therebetween. For purposes of this disclosure the term fluid should be defined to include any substance that continually deforms under an applied shear stress and may therefore include both liquids and gases. The pneumatic circuit connector 42 may comprise an annular retention shoulder 48 extending radially outward from the cylindrical body 44. The pneumatic circuit connector 42 may comprise a circumferential actuator 50 extending from the shoulder 48 and at least partially circumscribing the body 44. The circumferential actuator 50 comprises a tapered exterior surface 52. The tapered exterior surface 52 includes a proximal end 54 in contact with the shoulder 48 and a distal end 56. The tapered exterior surface 52 may taper in a generally constant manner such that the exterior diameter of the circumferential actuator 50 becomes smaller toward the distal end 56.

The anesthesia machine connector 32 includes an inlet port 60, an outlet port 62, an outlet 64 and a valve assembly 66. The inlet port 60 is adapted to receive fresh gas 29 (shown in FIG. 1) from the vaporizer 26 (shown in FIG. 1). The inlet port 60 may be pneumatically coupled with either the outlet port 62 or the outlet 64 depending on the position of the valve assembly 66. Fresh gas 29 from the outlet port 62 is delivered to the scavenging system 33 (shown in FIG. 1). Fresh gas 29 from the outlet 64 is delivered through the pneumatic circuit 30 to the patient 34 (shown in FIG. 1).

The valve assembly 66 includes a valve body 70, valve piston 72, a valve seal 74, and a valve spring 76. The valve body 70 may be generally cylindrical defining a hollow valve bore 78. The valve body 70 may define a first valve seat 80 and a second valve seat 82. The valve piston 72 may be axially translatable relative to the valve body 70 through the valve bore 78. The valve seal 74 may comprise an annular member secured to and extending radially outward from the valve piston 72. The valve seal 74 is preferably composed of a semi-rigid elastomeric material configured to deform into engagement with one of the valve seats 80-82 to establish a pneumatic seal. The valve spring 76 may configured to apply a force biasing the valve body 70 into a first position in which the valve seal 74 is engaged with the first valve seat 80.

The anesthesia machine connector 32 will be described in accordance with an embodiment as comprising an interface manifold 36. The interface manifold 36 may comprise a manifold housing 90, a manifold body 92, a manifold piston 94, and a manifold spring 96. The manifold housing 90 may comprise a generally hollow cylindrical configuration defining an exterior surface 98 and an interior surface 100. The exterior surface 98 may comprise a pivotable or hinged retention apparatus 102. The interior surface 100 may define a tapered geometry configured to compliment and accommodate the tapered exterior surface 52 of the circumferential actuator 50.

The manifold body 92 may be at least partially circumscribed by and axially translatable relative to the manifold housing 90. The manifold piston 94 may be at least partially circumscribed by and axially translatable relative to the manifold body 92. The manifold spring 96 may bias the manifold body 92 toward and into engagement with the pneumatic circuit connector 42 when the pneumatic circuit 30 is coupled with the anesthesia machine 10 (shown in FIG. 1).

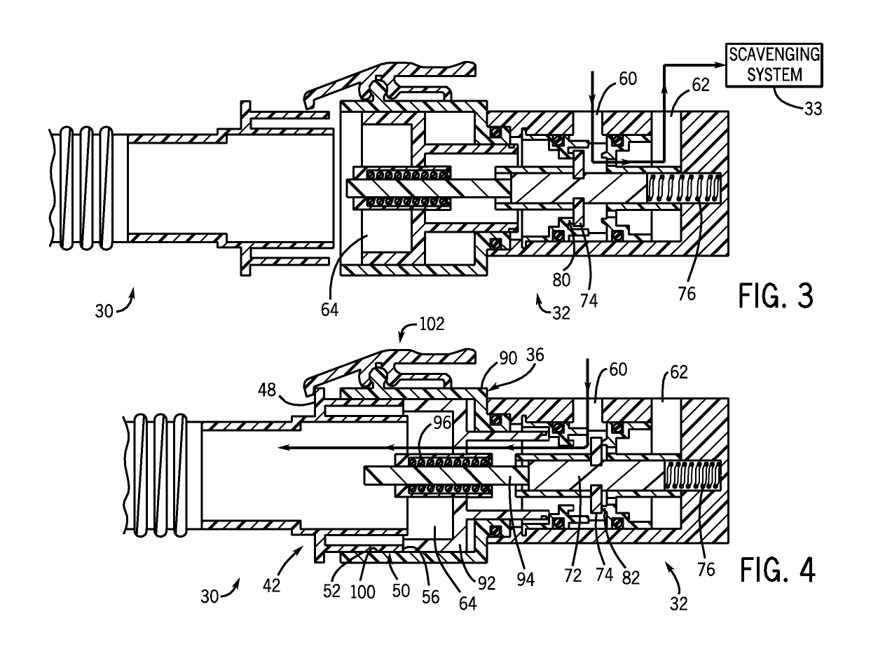

Having described the components of the pneumatic circuit 30 and the anesthesia machine connector 32 in accordance with an embodiment, their operation will now be described in more detail. Referring now to FIG. 3, the pneumatic circuit 30 is depicted as being disassembled from the anesthesia machine connector 32.

When the pneumatic circuit 30 is disassembled from the anesthesia machine connector 32, the anesthesia machine 10 (shown in FIG. 1) is operable in a first mode. In this first mode, the valve return spring 76 pushes the valve seal 74 into engagement with the first valve seat 80. When the valve seal 74 is engaged with the first valve seat 80, fresh gas 29 (shown in FIG. 1) from the inlet port 60 is precluded from passing through the outlet 64 to the patient 34 (shown in FIG. 1). This valve seal 74 position also has the effect of pneumatically coupling the inlet port 60 with the outlet port 62 such that the fresh gas 29 may be delivered as shown by the dashed line arrows through the inlet port 60, out the outlet port 62 and to the scavenging system 33. It should be appreciated that this first operational mode is the default for the anesthesia machine 10 (shown in FIG. 1), and that all of the fresh gas 29 may be delivered from the inlet port 60, through the outlet port 62 and to the scavenging system 33 until the pneumatic circuit 30 is connected.

Referring now to FIG. 4, the pneumatic circuit 30 is depicted as being assembled to the anesthesia machine connector 32. When the pneumatic circuit 30 is assembled to the anesthesia machine connector 32, the anesthesia machine 10 (shown in FIG. 1) is automatically converted to a second operational mode. In the second operational mode, fresh gas 29 (shown in FIG. 1) from the inlet port 60 is transferrable through the outlet 64 and to the patient 34 (shown in FIG. 1). The anesthesia machine 10 may be electrically converted to the second operational mode with a sensor (not shown) adapted to sense the connection of the pneumatic circuit 30. The anesthesia machine 10 may also be mechanically converted to the second operational mode as will now be described in detail.

As the pneumatic circuit connector 42 initially comes into contact with the anesthesia machine connector 32, the distal end 56 of the circumferential actuator 50 engages the manifold body 92. Additional insertion of the pneumatic circuit connector 42 pushes and translates the manifold body 92 and the manifold piston 94 in an inward direction. The manifold piston 94 is disposed in engagement with the valve piston 72 such that inward translation of the manifold piston 94 has the effect of inwardly translating the valve piston 72 and compressing the valve spring 76. Inward translation of the valve piston 72 pushes the valve seal 74 into engagement with the second valve seat 82. When the valve seal 74 is engaged with the second valve seat 82, fresh gas 29 (shown in FIG. 1) from the inlet port 60 is precluded from passing through the outlet port 62 to the scavenging system 33 (shown in FIG. 1). This valve seal 74 position also has the effect of pneumatically coupling the inlet port 60 with the outlet 64 such that the fresh gas 29 is delivered as shown by the dashed line arrow through the inlet port 60, through the outlet 64 and to the patient 34 (shown in FIG. 1).

After the valve seal 74 is brought into engagement with the second valve seat 82, additional insertion of the pneumatic circuit connector 42 has the effect of compressing the manifold spring 96. It should be appreciated that the manifold spring 96 is an optional component adapted to protect the valve seal 74 from damage attributable to over-insertion of the pneumatic circuit connector 42. As the manifold spring 96 is being compressed, the tapered exterior surface 52 of the circumferential actuator 50 may form a pneumatic seal with the complementary tapered interior surface 100 of the manifold housing 90. Alternatively, a pneumatic seal may be formed between the pneumatic circuit connector 42 and the anesthesia machine connector 32 in any known manner such as with an elastomeric O-ring seal (not shown). When the pneumatic circuit connector 42 is fully inserted into the anesthesia machine connector 32, the hinged retention apparatus 102 of the interface manifold 36 engages the annular retention shoulder 48 of the pneumatic circuit connector 42 to prevent unintentional decoupling.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

I claim:

1. An anesthesia machine comprising:
a gas mixer; and
an anesthesia machine connector pneumatically coupled with the gas mixer, said anesthesia machine connector adapted to selectively receive a pneumatic circuit;
wherein said anesthesia machine is operable in a first mode when the pneumatic circuit is decoupled from the anesthesia machine connector, said first mode comprising the transfer of a gas from the gas mixer to a first destination;
wherein said anesthesia machine is automatically converted to a second mode when the pneumatic circuit is coupled with the anesthesia machine connector, said second mode comprising the transfer of the gas from the gas mixer to a second destination distinct from said first destination.

2. The anesthesia machine of claim 1, wherein the first destination comprises a scavenging system.

3. The anesthesia machine of claim 1, wherein the second destination comprises a patient.

4. The anesthesia machine of claim 1, wherein the anesthesia machine is electrically converted to a second mode.

5. The anesthesia machine of claim 1, wherein the anesthesia machine is mechanically converted to a second mode.

6. The anesthesia machine of claim 1, wherein the anesthesia machine connector comprises a valve assembly.

7. The anesthesia machine of claim 6, wherein the anesthesia machine connector comprises an interface manifold adapted to protect the valve assembly.

8. An anesthesia machine comprising:
a gas mixer; and
an anesthesia machine connector pneumatically coupled with the gas mixer, said anesthesia machine connector adapted to selectively receive a pneumatic circuit, said anesthesia machine connector comprising:
an inlet port adapted to receive a gas from the gas mixer;
an outlet port selectively connectable with the inlet port, said outlet port adapted to direct the gas to a first destination;
an outlet selectively connectable with the inlet port, said outlet adapted to direct the gas to a second destination; and
a valve assembly adapted to pneumatically couple the inlet port with the outlet port or the outlet;
wherein the valve assembly couples the inlet port with the outlet port when the pneumatic circuit is disconnected from the anesthesia machine connector;
wherein the valve assembly automatically couples the inlet port with the outlet when the pneumatic circuit is connected to the anesthesia machine connector.

9. The anesthesia machine of claim 8, wherein the first destination comprises a scavenging system.

10. The anesthesia machine of claim 8, wherein the second destination comprises a patient.

11. The anesthesia machine of claim 8, wherein the anesthesia machine connector comprises an interface manifold adapted to protect the valve assembly.

12. An anesthesia system comprising:
a pneumatic circuit;
an anesthesia machine comprising:
a gas mixer; and
an anesthesia machine connector pneumatically coupled with the gas mixer, said anesthesia machine connector adapted to selectively receive the pneumatic circuit; and
a scavenging system pneumatically coupled with the anesthesia machine;
wherein said anesthesia machine is operable in a first mode when the pneumatic circuit is decoupled from the anesthesia machine connector, said first mode comprising the transfer of a gas from the gas mixer to the scavenging system;
wherein said anesthesia machine is automatically converted to a second mode when the pneumatic circuit is coupled with the anesthesia machine connector, said second mode comprising the transfer of the gas from the gas mixer to a patient.

13. The anesthesia system of claim 12, wherein the anesthesia machine connector comprises a valve assembly.

14. The anesthesia system of claim 13, wherein the anesthesia machine connector comprises an intake manifold adapted to protect the valve assembly.

15. A pneumatic circuit comprising:
a tube; and
a pneumatic circuit connector secured to the tube, said pneumatic circuit connector adapted to selectively couple with an anesthesia machine;
wherein said pneumatic circuit connector is configured to trigger a first anesthesia machine mode when the pneumatic circuit is disconnected from the anesthesia machine, said first anesthesia machine mode comprising the transfer of a gas to a first destination;
wherein said pneumatic circuit connector is configured to trigger a second anesthesia machine mode when the pneumatic circuit is connected to the anesthesia machine, said second anesthesia machine mode comprising the transfer of a gas to a second destination distinct from said first destination.

16. The pneumatic circuit of claim 15, wherein the first destination comprises a scavenging system.

17. The pneumatic circuit of claim 15, wherein the second destination comprises a patient.

18. The pneumatic circuit of claim 15, wherein the pneumatic circuit connector is configured to mechanically trigger the first and second anesthesia machine modes.
